SEQUENCE LISTING (15 sequences)

SEQ ID NO: 1 - DNA, 20 bp, Artificial Sequence
  A base sequence complementary to the base sequence (base sequence of SEQ ID NO 11 in the Sequence Listing) consisting of 20 bases containing the translational initiation codon AUG of PB2 gene of influenza virus.
ttctttccatattgaatata

SEQ ID NO: 2 - DNA, 20 bp, Artificial Sequence
  A base sequence complementary to the base sequence (base sequence of SEQ ID NO 11 in the Sequence Listing) consisting of 20 bases containing the translational initiation codon AUG of PB2 gene of influenza virus.
ttctttccatattgaatata

SEQ ID NO: 3 - DNA, 20 bp, Artificial Sequence
  A random base sequence with respect to the base sequence (base sequence of SEQ ID NO 11 in the Sequence Listing) consisting of 20 bases containing the translational initiation codon AUG of PB2 gene of influenza virus.
cttcttatatacgattatat

SEQ ID NO: 4 - DNA, 20 bp, Artificial Sequence
  A base sequence complementary to the base sequence (base sequence of SEQ ID NO 13 in the Sequence Listing) consisting of 20 bases containing the translational initiation codon AUG of PA gene of influenza virus.
aatcttccattttggatcag

SEQ ID NO: 5 - DNA, 20 bp, Artificial Sequence
  A base sequence complementary to the base sequence (base sequence of SEQ ID NO 13 in the Sequence Listing) consisting of 20 bases containing the translational initiation codon AUG of PA gene of influenza virus.
aatcttccattttggatcag

SEQ ID NO: 6 - DNA, 20 bp, Artificial Sequence
  A random base sequence with respect to the base sequence (base sequence of SEQ ID NO 13 in the Sequence Listing) consisting of 20 bases containing the translational initiation codon AUG of PA gene of influenza virus.
tcataagttcgactttcgta

SEQ ID NO: 7 - DNA, 28 bp, Artificial Sequence
  A base sequence complementary to the base sequence (base sequence of SEQ ID NO 12 in the Sequence Listing) consisting of 28 bases containing the translational initiation codon AUG of PB2 gene of influenza virus.
tttattctttccatattgaatataattg

SEQ ID NO: 8 - DNA, 28 bp, Artificial Sequence
  A base sequence complementary to the base sequence (base sequence of SEQ ID NO 12 in the Sequence Listing) consisting of 28 bases containing the translational initiation codon AUG of PB2 gene of influenza virus.
tttattctttccatattgaatataattg

SEQ ID NO: 9 - DNA, 28 bp, Artificial Sequence
  A base sequence complementary to the base sequence consisting of 28 bases containing the translational initiation codon AUG of PA gene of influenza virus.
acaaaatcttccattttggatcagtacc

SEQ ID NO: 10 - DNA, 28 bp, Artificial Sequence
  A base sequence complementary to the base sequence consisting of 28 bases containing the translational initiation codon AUG of PA gene of influenza virus.
acaaaatcttccattttggatcagtacc

SEQ ID NO: 11 - RNA, 20 nt, Influenza virus
uauauucaauauggaaagaa

SEQ ID NO: 12 - RNA, 28 nt, Influenza virus
caauuauauucaauauggaaagaauaaa

SEQ ID NO: 13 - RNA, 20 nt, Influenza virus
cugauccaaaauggaagauu

SEQ ID NO: 14 - RNA, 50 nt, Influenza virus
agcgaaagcaggucaauuauauucaauauggaaagaauaaaagaacuaag

SEQ ID NO: 15 - RNA, 54 nt, Influenza virus
agcgaaagcagguacugauccaaaauggaagauuuugugcgacaaugcuucaau

(19) United States
(12) Patent Application Publication
(10) Pub. No.: US 2003/0087851 A1
(43) Pub. Date: May 8, 2003

(54) Pharmaceutical composition for treating or preventing influenza, and novel oligonucleotide

(75) Inventors: Hiroshi Takaku, Chiba (JP); Kazuyuki Takai, Chiba (JP); Toshifumi Hatta, Ibaraki (JP); Tadashi Mizuta, Kanagawa (JP); Shiro Shigeta, Fukushima (JP); Tomoyuki Yokota, Fukushima (JP)

Correspondence Address: SUGHRUE MION ZINN,, MACPEAK & SEAS, PLLC, 2100 Pennsylvania Avenue, NW, Washington, DC, 20037-3213, US

(73) Assignee: BIOZAK, INC.

(21) Appl. No.: 09/907,666

(22) Filed: Jul. 19, 2001

Related U.S. Application Data
(63) Continuation-in-part of application No. PCT/JP99/00188, filed on Jan. 20, 1999.

(51) Int. Cl. A61K 48/00; A61K 9/127; C12N 15/88

(52) U.S. Cl. 514/44; 424/450; 435/458

(57) ABSTRACT

A pharmaceutical composition for treating or preventing influenza comprising an oligonucleotide containing a base sequence complementary to a base sequence of a target region containing a translational initiation codon AUG of a PB2 gene or a PA gene, a liposome stable in blood, and a pharmaceutically acceptable carrier or dilute is disclosed. The pharmaceutical composition for treating or preventing influenza can be used in an effective treatment of or prevention of an infection by influenza viruses.

### TECHNICAL FIELD

The present invention relates to a pharmaceutical composition for treating or preventing influenza, comprising an anti-influenza-viral antisense oligonucleotide and a liposome.

### BACKGROUND OF THE INVENTION

An influenza virus causes a severe cold with strong generalized symptoms. Particularly, in an aged patient or a high-risk patient suffering from a chronic respiratory disorder or a heart disease, influenza is a very infectious disease that often leads to a lethal pneumonia. The influenza viruses are classified into three types, A, B, and C, on the basis of the differences in serotypes of nucleoproteins (NP) and membrane proteins (M). Of these types, the influenza A virus and influenza B virus are prevalent every year. The influenza A virus has two glycoproteins, i.e., a hemaglutinin (HA) and a neuraminidase (NA), on the surface of an envelope thereof, and thus is classified into subtypes such as H1N1 (Soviet Union subtype), H2N2 (Asian subtype), and H3N2 (Hong Kong subtype), on the basis of the antigenecities thereof. The influenza B virus has both HA and NA, but there is only one subtype. Unlike the influenza A virus and the influenza B virus, the influenza C virus has only a hemaglutinin-esterase (HE) as a glycoprotein on the surface of the envelope, and thus, there is only one subtype.

The influenza virus belongs to the orthomyxoviridae family, and has a minus strand, i.e., a single-stranded RNA virus. The gene of the influenza virus is composed of eight segments. Proteins encoded by the eight segmentation genes include HA and NA, as well as M1 and M2, which are the membrane protein, on the surface of the envelope. Furthermore, a nucleoprotein complex (RNP) is located at the center of the virus and is composed of the RNA gene, three RNA polymerase subunits (PB1, PB2, and PA), and a nucleoprotein (NP). A non-structural protein (NS) is synthesized from the eighth segmentation gene.

The above three proteins PB1, PB2, and PA in the influenza virus are subunits which constitute an RNA polymerase. The RNA polymerase catalyzes a synthesizing reaction of RNA, an addition reaction of poly A, a restriction reaction of a cap, or the like. In particular, PB1 participates in the synthesizing reaction of RNA, and PB2 recognizes an mRNA cap structure of a host cell, and cleaves mRNA. PA has a role in transcription and elongation reactions. NP is a polynucleotide-binding protein non-specific to a base sequence. An RNA in an NP-RNA complex forms a double-stranded chain or a helix, and participates in a transcriptional reaction of a virus. Further, PB2, PB1, PA, NP, and NS are synthesized in an initial stage of the infection.

The influenza virus is initially adsorbed by a receptor on a cell membrane via HA. The receptor has sialic acid at a terminus thereof. Then the virus is incorporated into the cytoplasm by endocytosis. The stereostructure of the HA molecule is changed under acidic conditions in the endosome, and the HA molecule is cleaved to subunits HA1 and HA2 and activated by proteases in a host cell. As a result, a membrane fusion takes place between the virus envelope and the endosome membrane, and a virus gene is released into the cytoplasm to thereby generate an infection.

Generally, a human influenzal lesion is a local infection remaining at an upper portion of a respiratory tract, but does not proceed to pneumonia. Nevertheless, in an influenzal pneumonia of the high-risk patient, there are many cases wherein an expansion of a virally infectious focus is observed in pulmonary lesion. Further, such patients also suffer from a secondary bacterial pneumonia. The report of T. Akaike, et al., J. Virol. 63, 2252-2259 (1989) has shown that some bacteria present in a lung because of a mixed infection or a preceding infection produce a protease that facilitates a cleavage and activation of HA, and can directly activate the influenza virus, and has suggested the possibility of an enhancement of a proliferation and pathogenicity of the virus by the protease.

Of the influenza viruses, the influenza A virus undergoes a substantial change in antigenecity, and prevails every year above all others. In view of an infectivity thereof, the influenza A virus is most malignant. As an antiviral agent for the influenza A virus, amantadine and rimantadine are known, but they are not wholely satisfactory, because they cannot cope with mutants and have strong side effects. Further, a treatment by an inactivated vaccine has been attempted, but the vaccine cannot sustain a productivity of antibodies for a long period, and thus cannot completely prevent the spread of infection. Therefore, the development of a vaccine of an attenuated virus is desired so as to prolong the sustainability of the effects. Nevertheless, such a vaccine has not been developed. One reason for this is that a remarkable effect cannot be obtained by a vaccine because of the severity of an antigenic variation of the virus, and this is a cause of the delay in the development of the vaccine. As above, an effective method for treatment does not exist in the true sense of the term, but under the present situation, the prevention is intended by an administration of the vaccine, to some extent.

Recent research, developments, and investigations of novel substances exhibiting an anti-influenza virus activity have produced remarkable results. There are some candidate substances, for example, aprotinin, which is a protease inhibitor, inhibits a cleavage and activation of HA by a protease and thus prevents an infection; PM-523 inhibits membrane fusion and suppresses an invasion of the virus; BL-1743 inhibits the M2 ion channel and suppresses enucleation; L-735882 inhibits capping of mRNA and suppresses transcription; ribavirin inhibits RNA polymerase and suppresses transcription and replication; or GG-167 or GS4104 which inhibit neuraminidase and suppress a release. Pre-clinical tests or clinical tests are being conducted for these substances, which have shown a high potential; however, there are problems in regards to the generation of resistant viral strains, and strong side effects.

As an antiviral agent for the influenza viruses encountering very frequent mutations, it would be suitable to use a method wherein a gene is a target, e.g., an antisense oligonucleotide method. In the antisense oligonucleotide method, an oligonucleotide having a base sequence complementary to that of a target gene is used to inhibit a transcription, splicing, and/or translation of the target gene, at a DNA level or an mRNA level, and thus specifically prevent the expression of viral proteins [S. T. Crooke, Therapeutic Applications of Oligonucleotides, Springer-Verlag, (1995)]. Some important problems in the development of the antisense oligonucleotide method are the stability of the oligonucleotide in a body and an incorporation of the oligonucleotide into cells.

A method to conduct a direct transfection of genes into a body is, for example, a chemical method such as a method utilizing a precipitation by calcium phosphate, DEAE dextran, polyprene, polylysine, or a liposome; a physical method such as a microinjection or an electroporation; or a biological method such as a method utilizing a viral vector. Of these methods, the development of the methods utilizing the viral vector or the liposome is proceeding well. The method utilizing the viral vector has an advantage in a high efficiency of a viral transfection, but has a disadvantage in that it cannot be applied to a DNA having a large size. Further, there are problems of carcinogenicity, antigenecity, and cytotoxicity. On the other hand, the method utilizing the liposome has a disadvantage in a low efficiency of transfection, but is considered convenient because there is no limitation in the shape and size of a DNA for transfection, there is no risk of pathogenicity and antigenecity, and thus it is extremely safe.

Nevertheless, little has been reported of cases wherein the effectiveness of the antisense method is confirmed in an in vivo test. For the antisense method to the influenza viruses, there has been no report that confirms the effectiveness in an in vivo test.

The present inventors conducted investigations to develop an antisense method effective to the influenza viruses in in vivo experiments, and found that the above problems can be resolved by a combination of an antisense oligonucleotide to a particular target region in the influenza virus gene, and a particular liposome. The present invention is based on the above findings.

### DISCLOSURE OF INVENTION

The present invention relates to a pharmaceutical composition for treating or preventing influenza, comprising an oligonucleotide containing a base sequence complementary to a base sequence of a target region containing a translational initiation codon AUG of a PB2 gene or a PA gene, a liposome stable in blood, and a pharmaceutically acceptable carrier or dilute.

The present invention also relates to a method for treating or preventing influenza, comprising administering to a subject in need thereof an oligonucleotide containing a base sequence complementary to a base sequence of a target region containing a translational initiation codon AUG of a PB2 gene or a PA gene, a liposome stable in blood, in an amount effective in treating or preventing influenza.

The present invention also relates to a novel oligonucleotide containing a base sequence of SEQ ID NO: 8 or a base sequence of SEQ ID NO: 10. Each of the oligonucleotide is novel and effective for the above pharmaceutical composition for treating or preventing influenza.

The present invention also relates to a pharmaceutical composition comprising the oligonucleotide containing a base sequence of SEQ ID NO: 8 or a base sequence of SEQ ID NO: 10, and a pharmaceutically acceptable carrier or dilute.

The present invention also relates to a method for treating or preventing influenza, comprising administering to a subject in need thereof the oligonucleotide containing a base sequence of SEQ ID NO: 8 or a base sequence of SEQ ID NO: 10, in an amount effective in treating or preventing influenza.

### BRIEF DESCRIPTION OF DRAWINGS

FIG. 1 illustrates a partial base sequence of the PB2 gene of the influenza virus.

FIG. 2 illustrates a partial base sequence of the PA gene of the influenza virus.

FIG. 3 illustrates base sequences of the oligonucleotides according to the present invention and comparative oligonucleotides which are designed in Example 1.

FIG. 4 is a schedule of infections to mice and administrations of test solutions in experiments carried out in Example 3.

FIG. 5 is a graph showing a change in body weights of mice, caused by administering test solutions, in experiments carried out in Example 3.

FIG. 6 is a graph showing a change in body weights of mice, caused by administering other test solutions, in experiments carried out in Example 3.

FIG. 7 is a graph showing a change in body weights of mice, caused by administering still other test solutions, in experiments carried out in Example 3.

### BEST MODE FOR CARRYING OUT THE INVENTION

The present invention will be explained in detail hereinafter.

The term “influenza virus” as used herein includes the influenza A, the influenza B, and the influenza C, and the mutants thereof.

Therefore, for example, the “PB2 gene of the influenza virus” means each of PB2 genes (plus strands) of the influenza A virus, the influenza B virus, and the influenza C virus, and the mutants thereof. Similarly, the “PA gene of the influenza virus” means each of PA genes (plus strands) of the influenza A virus, the influenza B virus, and the influenza C virus, and the mutants thereof.

A target region of the oligonucleotide used in the pharmaceutical composition for treating or preventing influenza according to the present invention is a region containing a translational initiation codon AUG of the PB2 gene or the PA gene of the influenza virus, and thus, the oligonucleotide used in the pharmaceutical composition according to the present invention contains a base sequence complementary to a base sequence of the target region. The target region is not particularly limited so long as it contains the translational initiation codon AUG, and thus the target region may contain regions upstream and/or downstream of the translational initiation codon AUG.

The number of bases in the oligonucleotide used in the pharmaceutical composition for treating or preventing influenza according to the present invention is not particularly limited, but is preferably not less than the base number that allows a specific hybridization with the target region, and is preferably not more than the base number that allows a penetration of the oligonucleotide used in the present invention through a cell or a nuclear membrane.

The base number that allows a specific hybridization with the target region is preferably 15 bases or more, more preferably 20 bases or more. The base number that ensures the membrane penetration is preferably 30 bases or less, more preferably 28 bases or less. The oligonucleotide used in the present invention consists of preferably 15 to 30 bases, more preferably 20 to 28 bases.

The oligonucleotide used in the present invention need not contain a contiguous base sequence complementary to that of the target region so long as it can specifically hybridize the target region (for example, an mRNA) to form a double-stranded chain. In the oligonucleotide, one or more non-complementary bases may be deleted, inserted and/or substituted at one or more positions. However, the oligonucleotide preferably contains the contiguous base sequence complementary to that of the target region.

The concrete base sequence may be suitably determined in accordance with the type of the target influenza virus. For example, when the target is a mutant of the influenza A virus, a base sequence of a region containing a translational initiation codon AUG of the PB2 gene and/or the PA gene of the mutant is analyzed. Then, the number of the bases and the target sequence region are determined taking into account that a stable double-stranded chain may be formed, and an antisense oligonucleotide may be synthesized by a known method.

In the oligonucleotide used in the present invention, internucleotide bonds between nucleotides may be independently a phosphodiester bond or a modified phosphodiester bond. The modified phosphodiester may be, for example, a methylphosphonate type bond wherein one of two non-crosslinked oxygen atoms in the phosphodiester bond is replaced with a methyl group; a phosphoroamidate type bond wherein one of two non-crosslinked oxygen atoms in the phosphodiester bond is replaced with an amino group or a substituted amino group; a phosphorothioate type bond wherein one of two non-crosslinked oxygen atoms in the phosphodiester bond is replaced with a sulfur atom; or a phosphorodithioate type bond wherein each of two non-crosslinked oxygen atoms in the phosphodiester bond is replaced with a sulfur atom. The oligonucleotide may contain one or more modified phosphodiester bonds as above in one or more internucleotide bonds. The modified phosphodiester bond is preferable, from the standpoints of the specificity to a base sequence, a stability of the double-stranded chain, a resistance to a nuclease, a penetrating property through a cell membrane, a low cytotoxicity, a moderate metabolizability, an easy procedure for preparation, and so on. Further, the phosphorothioate type bond is more preferable from the standpoint of a stability in a living body. It is particularly preferable that not less than half, or in particular all, of the internucleotide bonds are the modified phosphodiester bonds, in particular the phosphorothioate type bonds.

In the oligonucleotide used in the present invention, thymine (T) or uracil (U), cytosine (C), guanine (G), and adenine (A) may be used as the base complementary to adenine (A), guanine (G), cytosine (C), and uracil (U), which are the constituent bases of the base sequence of the target gene in the influenza virus. When the oligonucleotide used in the present invention is an RNA, U is used as the base complementary to A constituting the base sequence of the target gene in the influenza virus.

The oligonucleotide used in the present invention may be prepared from deoxyribonucleosides, ribonucleosides, and/or modified ribonucleosides thereof, such as 2′-O-modified ribonucleosides, so long as the resulting oligonucleotide can specifically hybridize the target region to form a stable double-stranded chain. A preferable modified ribonucleoside is 2′-O-methylribonucleoside, in view of a strong binding property with the base sequence of the target. Therefore, the oligonucleotide used in the present invention may be an oligoribonucleotide (RNA) composed of ribonucleosides and/or modified ribonucleosides, an oligodeoxyribonucleotide (DNA) composed only of deoxyribonucleosides, or a chimera oligoribo/deoxyribonucleotide (RNA/DNA) composed of ribonucleosides (and/or modified ribonucleosides) and deoxyribonucleosides.

The oligonucleotide used in the present invention is, for example, an antisense oligonucleotide containing a base sequence complementary to a base sequence of a target region containing a translational initiation codon AUG of the PB2 gene and/or the PA gene of influenza virus A/PR/8/34 [PB1: G. Winter and S. Fields, Nucleic Acids Res. 10, 2135 (1982); PB2: G. Winter and S. Fields, Cell, 28, 203 (1982); NP: G. Winter and S. Fields, Viology, 114, 423 (1981)]. The antisense oligonucleotide is effective particularly against the influenza A virus. However, the antisense oligonucleotide is also effective against the influenza B virus and the influenza C virus.

As the antisense oligonucleotide containing a base sequence complementary to a base sequence of a region containing a translational initiation codon AUG of the PB2 gene, there may be mentioned, for example, an oligonucleotide containing at least 15 bases, preferably 15 to 30 bases, of the base sequence complementary to the base sequence of SEQ ID NO: 11. As the antisense oligonucleotide containing a base sequence complementary to a base sequence of a region containing a translational initiation codon AUG of the PA gene, there may be mentioned, for example, an oligonucleotide containing at least 15 bases, preferably 15 to 30 bases, of the base sequence complementary to the base sequence of SEQ ID NO: 13.

Specifically, the antisense oligonucleotide used in the present invention may be an oligonucleotide that consists of a base sequence complementary to a base sequence consisting of 20 bases and containing a translational initiation codon AUG of the PB2 gene, has phosphodiester bonds as internucleotide bonds, and has the base sequence of SEQ ID NO: 1; an oligonucleotide the same as the above oligonucleotide except that the internucleotide bonds are phosphorothioate bonds, and the base sequence is SEQ ID NO: 2; an oligonucleotide that consists of a base sequence complementary to a base sequence consisting of 28 bases and containing a translational initiation codon AUG of the PB2 gene, has phosphodiester bonds as internucleotide bonds, and has the base sequence of SEQ ID NO: 7; an oligonucleotide the same as the above oligonucleotide except that the internucleotide bonds are phosphorothioate bonds, and the base sequence is SEQ ID NO: 8; an oligonucleotide that consists of a base sequence complementary to a base sequence consisting of 20 bases and containing a translational initiation codon AUG of the PA gene, has phosphodiester bonds as internucleotide bonds, and has the base sequence of SEQ ID NO: 4; an oligonucleotide the same as the above oligonucleotide except that the internucleotide bonds are phosphorothioate bonds, and the base sequence is SEQ ID NO: 5; an oligonucleotide that consists of a base sequence complementary to a base sequence consisting of 28 bases and containing a translational initiation codon AUG of the PA gene, has phosphodiester bonds as internucleotide bonds, and has the base sequence of SEQ ID NO: 9; an oligonucleotide the same as the above oligonucleotide except that the internucleotide bonds are phosphorothioate bonds, and the base sequence is SEQ ID NO: 10. The eight oligonucleotides as above are effective particularly against the influenza A virus, respectively. Oligonucleotides complementary to the influenza B virus and the influenza C virus are also effective against the influenza B virus and the influenza C virus.

The oligonucleotide used in the present invention may be prepared by known methods. For example, the oligonucleotide may be prepared by an automated DNA/RNA synthesizer in accordance with a conventional phosphodiester method, or phosphotriester method, such as an H-phosphonate method or a phosphoramidite method, except for a site to which a 2′-O-methylribonucleotide or a phosphorothioate bond is introduced.

The oligonucleotide having 2′-O-methylribonucleotides may be prepared, for example, by an automated DNA/RNA synthesizer in accordance with the phosphoramidite method, using a 5′-dimethoxytrityl-2′-O-methylribonucleoside-3′-[(2-cyanoethyl)-(N,N-diisopropyl)]-phosphoroamidite unit.

The oligonucleotide having phosphorothioate bonds may be prepared, for example, using a 15% N,N,N′,N′-tetraethylthiorumdisulfide/acetonitrile solution instead of a water/iodine/pyridine solution that is an oxidizing agent used in a conventional synthesis of polynucleotide.

The liposome which may be used in the present invention is not particularly limited so long as it is stable in blood. The term “stable in blood” as used herein means that when the anti-viral agent of the present invention is administered to an animal, a deliverability of the oligonucleotide is maintained until it is delivered to an animal cell. The stability may be determined by examining an ability to take up an oligonucleotide into an animal cell after the anti-viral agent of the present invention is brought into contact with an animal serum for 24 to 72 hours, the whole is incubated at 27° C., and then an animal cell is added, or after the anti-viral agent of the present invention is incubated with an animal serum and an animal cell under the same conditions as above.

The liposome which may be used in the present invention is, for example, a liposome prepared from phospholipid, glycolipid, or lipid molecule, such as cholesterol. An unilamellar liposome or a multilamellar liposome may be effectively used.

As the phospholipid from which the liposome can be prepared, there may be mentioned, for example, glycerophospholipid (phosphatidylcholine, phosphatidylethanolamine, phosphatidylserine, phosphatidic acid, phosphatidylglycerol, phosphatidylinositol, or cardiolipin), or sphingophospholipid (sphingomyelin, ceramide phosphorylethanolamine, or ceramide phosphorylglycerol). As the glycolipid from which the liposome can be prepared, there may be mentioned, for example, glyceroglycolipid (digalactosyldiglyceride, or seminolipid), or sphingoglycolipid (galactosylceramide, or lactosylceramide).

In the present invention, commercially available liposomes may be used. Examples of the commercially available liposomes which may be used in the present invention are a mixture (Wako Pure Chemical Industries, Ltd.) of Genetransfer [composed of N-(α-trimethylammonioacetyl)dodecyl-D-glutamate chloride, L-(α-phosphatidylethanolamine dioleyl, and dilauryl-L-α-phosphatidylcholine at a ratio of 1:2:3] and HMG-1,2, a liposome (Tfx-10; Promega K. K.) prepared from a mixture of N,N,N′,N′-tetramethyl-N,N′-bis(2-hydroxyethyl)-2,3-dioleoyloxy-1,4-butanediammonium iodide and L-dioleoylphosphatydylethanolamine, a positively charged liposome (COATSOME EL-C-01; NOF Corporation) prepared from a mixture of L-α-dipalmitoylphosphatydylcholine, cholesterol, and stearylamine (52:40:8), or a weak negatively-charged liposome (COATSOME EL-N-01; NOF Corporation) prepared from a mixture of L-α-dipalmitoylphosphatydylcholine, cholesterol, and L-α-dipalmitoylphosphatydylglycerol (54:40:6).

From a charged state, liposome is classified into a neutral liposome, a negatively-charged liposome, a positively-charged liposome, a pH-sensitive liposome, or the like. The negatively-charged liposome, or the positively-charged liposome is preferable.

Particularly preferable liposomes in the present invention are a liposome (Tfx-10) prepared from a mixture of N,N,N′,N′-tetramethyl-N,N′-bis(2-hydroxyethyl)-2,3-dioleoyloxy-1,4-butanediammonium iodide and L-dioleoylphosphatydylethanolamine, or a liposome prepared from a mixture of L-α-dipalmitoylphosphatydylcholine, cholesterol, and stearylamine or L-α-dipalmitoylphosphatydylglycerol; or particularly, a positively-charged liposome (COATSOME EL-C-01) prepared from a mixture of L-α-dipalmitoylphosphatydylcholine, cholesterol, and stearylamine (52:40:8), or a weak negatively-charged liposome (COATSOME EL-N-01) prepared from a mixture of L-α-dipalmitoylphosphatydylcholine, cholesterol, and L-α-dipalmitoylphosphatydylglycerol (54:40:6).

The formulation of the pharmaceutical composition for treating or preventing influenza according to the present invention is not particularly limited, so long as the oligonucleotide and the liposome stable in blood are contained in the pharmaceutical composition. For example, the pharmaceutical composition may be a mixture or a complex of the oligonucleotide and the liposome, or a formulation prepared by embedding the oligonucleotide in the liposome or encapsulating the oligonucleotide with the liposome.

The complex may be prepared, for example, in accordance with a method for preparing a complex from the oligonucleotide and the liposome by making use of an electrostatic binding, that is, a method called a lipofection method, by slowly mixing the oligonucleotide and the liposome in a test tube and allowing to stand at room temperature for about 15 minutes.

The embedded formulation may be prepared, for example, in accordance with a method for embedding the oligonucleotide in the liposome. More particularly, a lipid such as phosphatidylserine is treated by a vortex mixer to produce a multilayered liposome. Then, the multilayered liposome is treated with ultrasonics to prepared an unilamellar liposome. The oligonucleotide is added to the resulting unilamellar liposome, and the whole is treated in a vortex mixer and incubated for about 10 minutes at about 37° C. or lyophilized and then re-hydrated. The encapsulated formulation may also be prepared by known methods.

The pharmaceutical composition for treating or preventing influenza according to the present invention may be administered via any of an oral, parenteral or local route. The dose may vary with the species of the subject to be treated (a mammal, particularly a human), a response of the subject to the medicine, a formulation of the medicine, an administration time, an interval of administrations, or the like, but may generally be about 500 mg to about 5000 mg/day.

The pharmaceutical composition for treating or preventing influenza according to the present invention may be administered in the form of a combination of the oligonucleotide and the liposome, and optionally, a pharmaceutically acceptable known carrier or dilute via any of the oral, parenteral or local routes, once or a multiple of times. The pharmaceutical composition for treating or preventing influenza according to the present invention may be variously formulated to produce, for example, tablets, capsules, lozenges, troches, hard candies, powders, sprays, creams, ointments, suppositories, jellies, gels, pastes, lotions, salves, aqueous suspensions, solutions for injection, elixirs, syrups, or the like.

The action of pharmaceutical composition for treating or preventing influenza according to the present invention is presumed, but by no means limited to the following presumption, for example, that if the antisense oligonucleotide is delivered from blood to an infected cell at an upper portion of a respiratory tract or a lung tissue with the aid of an enhanced membrane-penetrating property of a positively-charged liposome, the antisense oligonucleotide is bound to, for example, a replicated +cRNA to thereby inhibit a synthesis of a daughter viral RNA from the cRNA as a template. Further, it is presumed that the antisense oligonucleotide is bound to an mRNA transcripted from −vRNA which had been released from a nucleus of the infected cell to a cytoplasm. Therefore, an anti-viral effect against the influenza viruses can be synergistically enhanced by combining the antisense oligonucleotide and the liposome, and intravenously administering the same, for example, successively.

### EXAMPLES

The present invention now will be further illustrated by, but is by no means limited to, the following Examples.

### Example 1

### Design of Oligonucleotides

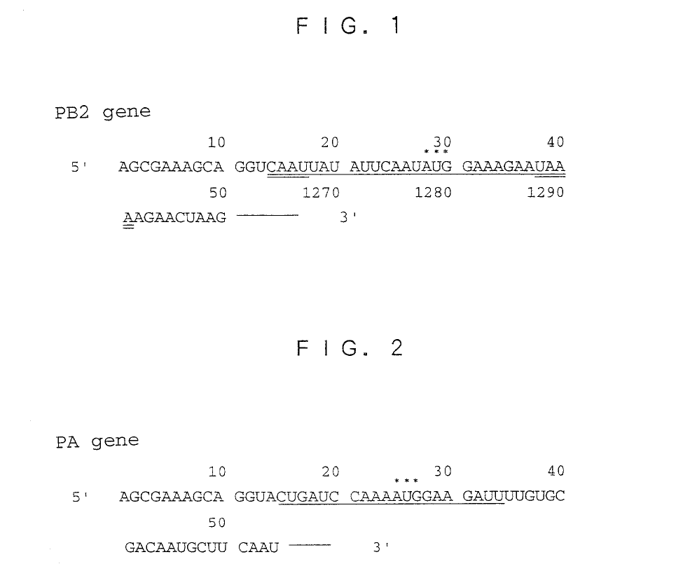

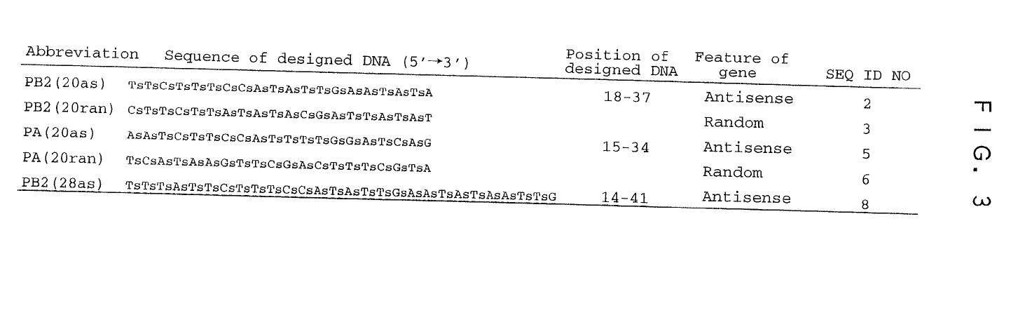

As the target genes for use in the following Examples, the PB2 gene and the PA gene were selected, and the translational initiation regions were selected. The base sequence in the translational initiation region of the PB2 gene is shown in FIG. 1, and the base sequence in the translational initiation region of the PA gene is shown in FIG. 2.

More particularly, two base sequences were selected from the translational initiation region of the PB2 gene. The first base sequence is the sequence from the 18th to 37th bases underlined in FIG. 1, and composed of 20 bases (the base sequence of SEQ ID NO: 11). The second base sequence is the sequence from the 14th to 41st bases containing each of 4 bases underlined twice in FIG. 1 in addition to the first base sequence upstream and downstream thereof, and composed of 28 bases (the base sequence of SEQ ID NO: 12). As the translational initiation region of the PA gene, the sequence from the 15th to 34th bases underlined in FIG. 2 (the base sequence of SEQ ID NO: 13) was selected. In FIGS. 1 and 2, the “AUG” marked *** denotes the translational initiation codon, and the mark “−” denotes an omitted base sequence.

Base sequences around the selected base sequences are also shown in FIGS. 1 and 2. More particularly, FIG. 1 shows the sequence from the 1st to 50th bases (the base sequence of SEQ ID NO: 14) containing the base sequence selected as the translational initiation region of the PB2 gene and the base sequences around it, and FIG. 2 shows the sequence from the 1st to 54th bases (the base sequence of SEQ ID NO: 15) containing the base sequence selected as the translational initiation region of the PA gene and the base sequences around it.

For the translational initiation regions of the PB2 gene and the PA gene, antisense oligonucleotides and random oligonucleotides were designed. The term “random oligonucleotide” as used herein means a base sequence designed so that the number of the bases (A, G, C, and T) constituting the random oligonucleotide is equal to the number of the bases constituting the corresponding antisense oligonucleotide, and the random oligonucleotide cannot form a double-stranded chain with any portion of the whole genes of the influenza viruses.

Further, oligonucleotides wherein all the internucleotide bonds were phosphorothioate type bonds were designed for each oligonucleotide of the translational initiation regions of the PB2 gene and the PA gene. Such an oligonucleotide will be hereinafter referred to as S-oligo.

The designed oligonucleotides are shown in FIG. 3. Symbols in the Abbreviation column of FIG. 3 have the following meanings: “20” and “28” mean that the number of the bases of the oligonucleotide is 20 or 28, respectively; “as” means that the designed DNA is an antisense against the selected sequence of the virus gene; and “ran” means that the designed DNA is random against the selected sequence of the virus gene. In the sequences of designed DNAs in FIG. 3, “A”, “G”, “C”, and “U” denote adenosine, guanosine, cytidine, and uridine, and “s” means that an internucleotide bond is a phosphorothioate type bond. In the “Position of designed DNA” column in FIG. 3, a numerical figure denotes a base number showing the location of the designed sequence in the virus gene as shown in FIGS. 1 and 2.

### Example 2

### Synthesis of oligonucleotides

Five oligonucleotides designed in Example 1 were synthesized, using an automated DNA synthesizer (Model 392: Applied Biosystems) in accordance with a program for a phosphorothioate type oligonucleotide. That is, oligonucleotides were synthesized in accordance with a phosphoramidite method using a solid phase column (1 μmol scale; Cruachem, United Kingdom) and reagents for DNA synthesis (Cruachem, United Kingdom), and then cut from the column and deprotected in accordance with the conventional method [A. Chollet & E. H. Kawashima, Nucleic Acids Res., 13, 1529 (1985)]. An amount of 1/500 (about 4 μg) of each of the resulting oligonucleotides was applied to a 20% polyacrylamide gel electrophoresis containing 7 M urea. The electrophoresis was carried out at a constant voltage of 150 V for 1.5 hours. After the electrophoresis was completed, the gel was stained with methylene blue to confirm that each of the synthesized oligonucleotides had a predetermined strand length.

For the confirmed oligonucleotides, an amount of ⅕ to ½ (about 1.2 mg) of the remaining oligonucleotides was subjected to the 20% polyacrylamide gel electrophoresis containing 7 M urea, in which electrophoresis was used for cutting out and purification. The electrophoresis was carried out at a constant voltage of 200 V for 6 hours. After the electrophoresis was completed, the gel was removed from the gel plate, covered with a wrap, and irradiated with ultraviolet light. Bands having the predetermined strand length were marked, and the marked gels were then divided into small pieces with a sterilized cutter, and collected into a 1.5 ml volume of sample tubes. To the tubes, 0.4 ml of a 10 mM Tris-HCl (pH 7.5)/1 mM-EDTA solution was added, and then DNAs were extracted from the gel pieces to the solutions with shaking at 37° C. for 3 to 12 hours. The extracts were collected, and extracted with phenol/chloroform to remove acrylamide. Thereafter, an ethanol precipitation was carried out to purify the oligonucleotides. The yields of the oligonucleotides were 0.3 to 0.6 mg. Each of the resulting oligonucleotides (about 0.5 μg) was applied to the polyacrylamide gel electrophoresis containing 7 M urea, and after the electrophoresis was completed, the gel was stained with ethidium bromide. Each of the oligonucleotides showed a single band.

### Example 3

### Successive Intravenous Administration of Antisense Oligonucleotides (1)

Five oligonucleotides prepared in Example 2, i.e., PB2 (20as), PB2 (20ran), PA (20as), PA (20ran), and PB2 (28as), were incubated at room temperature for 20 to 30 minutes in a sterilized phosphate buffered saline (PBS), a Tfx-10 solution in PBS (in an amount such that a dose of Tfx-10 is 5 mg/kg or less), a COATSOME EL-C-01 (NOF Corporation) solution in PBS (in an amount such that a dose of COATSOME EL-C-01 is 20 μmol/kg), and a COATSOME EL-N-01 solution in PBS (in an amount such that a dose of COATSOME EL-N-01 is 20 μmol/kg), and dissolved therein to prepare sample solutions containing the above oligonucleotides at a concentration of 0.2 (w/v) % or 0.4 (w/v) %. The sample solutions were stored in a refrigerator at 4° C., until used for a treatment of test animals (mice).

Female BALB/c mice (body weight=17 to 20 g; 6 weeks old) were used as the test animals, and divided into test groups. Each test group contained 6 to 11 mice. Each group of 6 to 11 mice was put into a mouse cage (width=22 cm, length=32 cm, height=13.5 cm) made of polycarbonate resin and equipped with stainless steel nets, and bred in a barrier breeding room for infected animals, at a temperature of 23±2° C. and a humidity of 55 ±10%, under a lighting time of 12 hours (from 7:00 to 19:00). Mice were able to freely take sterilized solid feed (MM-6; Funabashi Nojo) and tap water.

As an influenza A virus for an infection to mice, A/PR/8/34 strain (H1N1: hereinafter referred to as PR8) proliferated in a fertilized egg of a chicken was used. For a nasal inoculation to mice, a virus liquid having an LD₅₀ of 100 was prepared. Mice were anesthetized by intraperitoneally administering 50 mg/kg sodium pentobarbital, and nasally inoculated with the PB8 virus liquid at an amount of 40 μl/body to obtain infected mice.

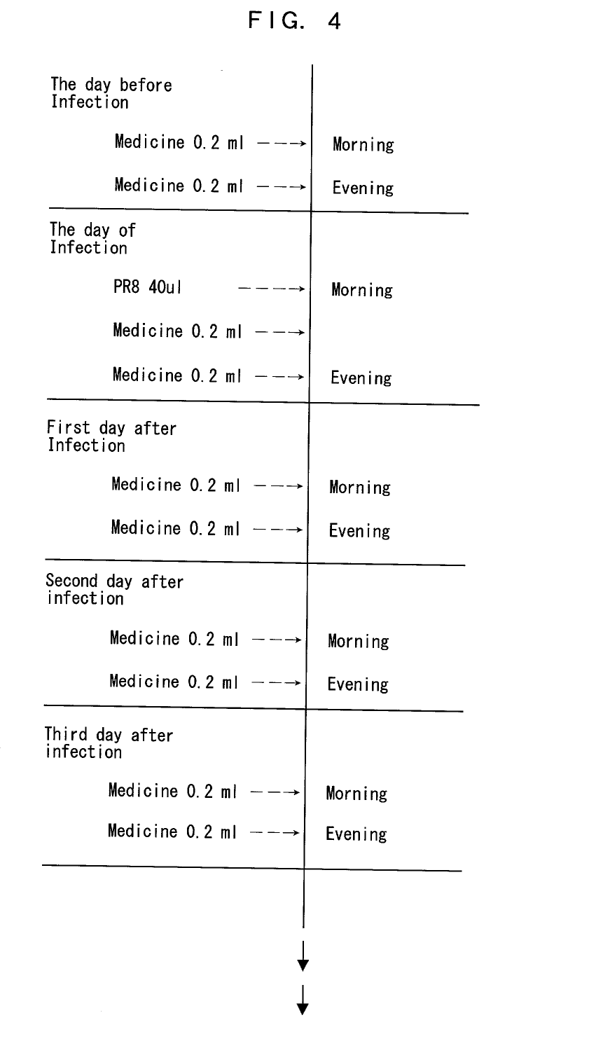

FIG. 4 shows an administration schedule of the test solutions and the infection to mice. The test solution was intravenously administered at a tail under an anesthetic every morning and evening from the day before the nasal inoculation to the 7th day after the nasal inoculation, at an amount of 0.2 ml/body weight. After the administration of the test solutions was finished, symptoms were generally observed every day for a further seven days, and body weights and death were recorded every evening. All of the dead mice and surviving mice at the 10th day after infection were examined by autopsy. Lungs were taken and homogenized by a glass-Teflon homogenizer. The resulting supernatant and monolayered MDCK cells were incubated for 4 days in a CO₂ incubator at 35° C., and then a virus titer in the lung was measured.

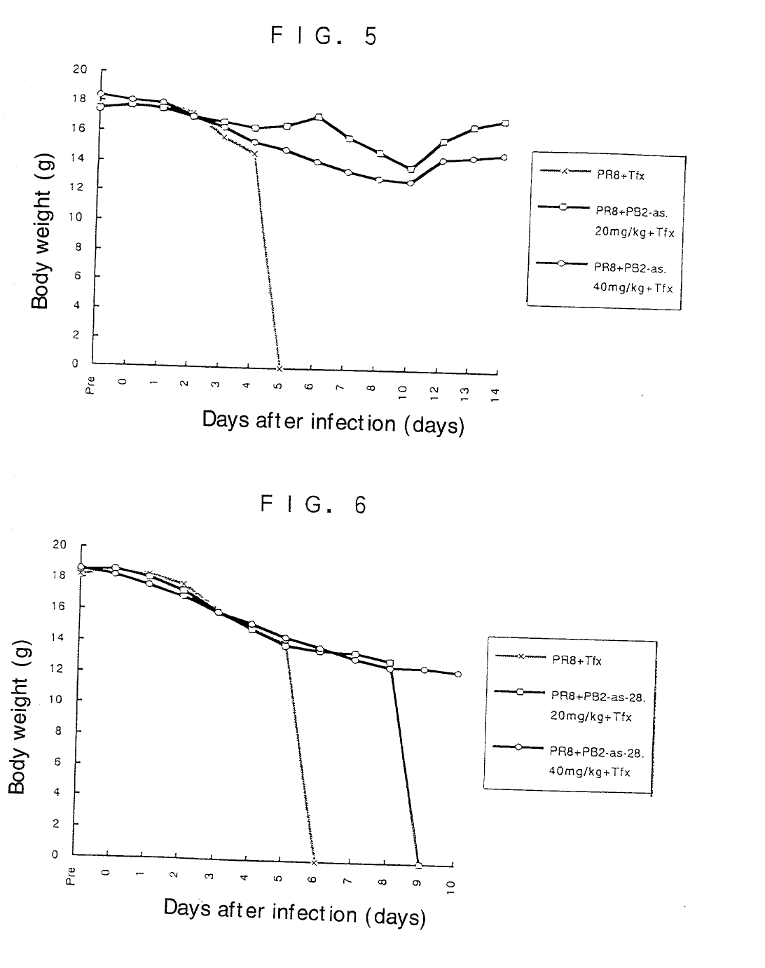

Changes in body weights with the administration of the sample solutions are shown in FIGS. 5 to 7. FIG. 5 shows the results obtained by administering the oligonucleotide PB2 (20as) and the Tfx-10 solution in PBS (dose=1 mg/kg).

As shown in FIG. 5, body weights of mice in the virus control group [no PB2 (20as) administered; a curve X in FIG. 5] began to decrease and fur was remarkably raised from about the second or third day after the inoculation with PR8. A remarkable decrease in the body weight continued to the point of death, and no mouse survived after the 5th day. The autopsy examination revealed that severe blood humectation in the lung was observed in all of the mice, and the virus titer was 10^{7.6} TCID₅₀/g lung weight. On the contrary, in the group to which PB2 (20as) was administered at an amount of 20 mg/kg with 1 mg/kg of Tfx-10 [a curve □ in FIG. 5], and the group to which PB2 (20as) was administered at an amount of 40 mg/kg with 1 mg/kg of Tfx-10 [a curve ◯ in FIG. 5], some mice survived after the 5th day, and the body weights were recovered in the surviving mice. The autopsy examination of the recovered cases revealed that no abnormality was observed in all cases. The virus titer was lowered to 10^{5.5} TCID₅₀/g lung weight, and the proliferation of the viruses was remarkably inhibited.

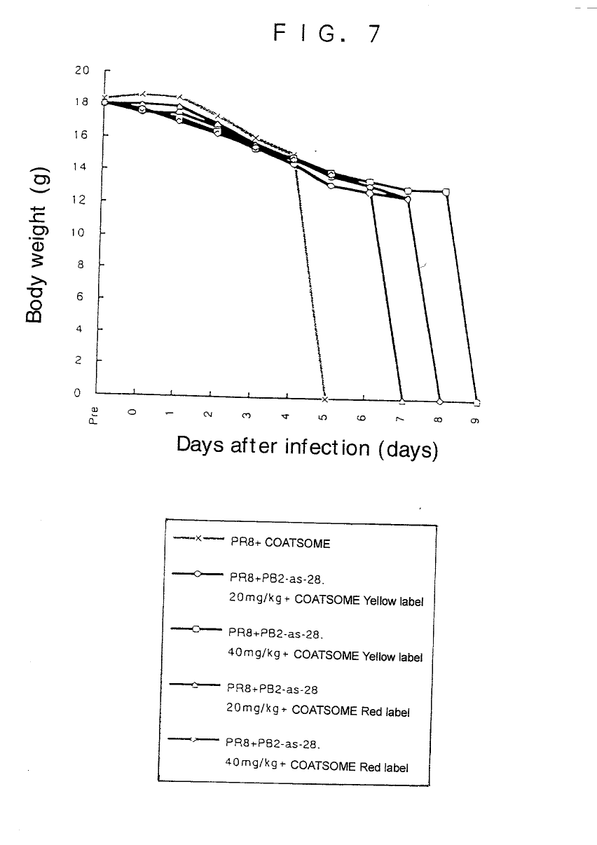

FIG. 6 shows the results obtained by administering the oligonucleotide PB2 (28as) and the Tfx-10 solution in PBS (dose=1 mg/kg).

In the virus control group [a curve X in FIG. 6], no mouse survived after the 6th day. In the group to which PB2 (28as) was administered at an amount of 20 mg/kg with 1 mg/kg of Tfx-10 [a curve □ in FIG. 6], the day when all of the mice had died was extended to the 9th day after the PR8 inoculation. Further, in the group to which PB2 (28as) was administered at an amount of 40 mg/kg with 1 mg/kg of Tfx-10 [a curve ◯ in FIG. 6], some mice still survived to the 10th day after the PR8 inoculation.

FIG. 7 shows the results obtained by administering the oligonucleotide PB2 (28as) and a COATSOME EL-C-01 (20 μmol/kg) or COATSOME EL-N-01 (20 μmol/kg) solution in PBS.

In the virus control group [a curve X in FIG. 7], no mouse survived on the 5th day after the PR8 inoculation. In the group to which PB2 (28as) was administered at an amount of 20 mg/kg with 20 μmol/kg of COATSOME EL-N-01 [a curve ◯ in FIG. 7], the day when all of the mice had died was extended to the 8th day after the PR8 inoculation, and in the group to which PB2 (28as) was administered at an amount of 40 mg/kg with 20 μmol/kg of COATSOME EL-N-01 [a curve □ in FIG. 7], the day when all of the mice had died was extended to the 9th day after the PR8 inoculation. Further, in the group to which PB2 (28as) was administered at an amount of 20 mg/kg with 20 μmol/kg of COATSOME EL-C-01 [a curve Δ in FIG. 7], the day when all of the mice had died was extended to the 7th day after the PR8 inoculation, and in the group to which PB2 (28as) was administered at an amount of 40 mg/kg with 20 μmol/kg of COATSOME EL-C-01 [a curve ⋄ in FIG. 7], the day when all of the mice had died was extended to the 8th day after the PR8 inoculation. The autopsy examination of the dead mice revealed that a severe blood humectation in the lung was observed in all of the mice, and the virus titer was 10^{6.0} TCID₅₀/g lung weight.

Table 1 shows changes of the survival rate with time in mice used in the above experiments.

In the control group wherein only Tfx-10 (5 mg/kg) was intravenously administered to the mice, all of the mice survived until the 10th day. Further, in the virus control group wherein Tfx-10 (5 mg/kg) was administered to the infected mice, 30% of the mice survived to the 5th day after the PR8 inoculation, but all of the mice were dead on the 6th day.

On the contrary, in the administration groups wherein PB (20as) was administered at an amount of 20 mg/kg with 1 mg/kg or 5 mg/kg of Tfx-10, 63% and 100% of the mice survived to the 6th day, respectively, about 50% to the 7th day, respectively, and about 25% or more from the 8th day to the 10th day. A significant effect on the prolonging of life was observed. No significant difference was observed in the dose of Tfx-10 between 1 mg/kg and 5 mg/kg.

TABLE 1

| | Survival rate (%) | | | | | | |
|---|---|---|---|---|---|---|---|
| | Number | | | | | | |
| Treatment | of mice | 5th day | 6th day | 7th day | 8th day | 9th day | 10th day |
| i.v. (control) | 8 | 100 | 100 | 100 | 100 | 100 | 100 |
| PR8 + Tfx 5 mg/kg i.v. (Virus control) | 10 | 30 | 0 | 0 | 0 | 0 | 0 |
| PR8 + PB2 (20 as) 20 mg/kg + Tfx 1 mg/kg i.v. | 8 | 88** | 63* | 50* | 38* | 38* | 25* |
| PR8 + PB2 (20 as) 40 mg/kg + Tfx 1 mg/kg i.v. | 8 | 100** | 100** | 75** | 75** | 50* | 25* |
| PR8 + PA (20 as) 20 mg/kg + Tfx 1 mg/kg i.v. | 8 | 0 | 0 | 0 | 0 | 0 | 0 |
| PR8 + PB2 (20 as) 20 mg/kg + Tfx 5 mg/kg i.v. | 11 | 100** | 100** | 54* | 27* | 27* | 27* |
| PR8 + PB2 (20 ran) 20 mg/kg + Tfx 5 mg/kg i.v. | 6 | 17 | 0 | 0 | 0 | 0 | 0 |
| PR8 + PA (20 as) 20 mg/kg + Tfx 5 mg/kg i.v. | 11 | 73* | 45* | 9 | 9 | 9 | 9 |
| PR8 + PA (20 ran) 20 mg/kg + Tfx 5 mg/kg i.v. | 6 | 0 | 0 | 0 | 0 | 0 | 0 |
| PR8 + PB2 (20 as) 40 mg/kg i.v. | 6 | 33 | 33* | 17 | 0 | 0 | 0 |
| PR8 + PA (20 as) 40 mg/kg i.v. | 6 | 33 | 17 | 0 | 0 | 0 | 0 |
| PB2 (20 as) 20 mg/kg + Tfx 5 mg/kg i.v. | 8 | 100 | 100 | 100 | 100 | 100 | 100 |
| PA (20 as) 20 mg/kg + Tfx 5 mg/kg i.v. | 8 | 100 | 100 | 100 | 100 | 100 | 100 |
| PR8 + PB2 (28 as) 20 mg/kg + Tfx 1 mg/kg i.v. | 7 | 86* | 57* | 29* | 29* | 0 | |
| PR8 + PB2 (28 as) 40 mg/kg + Tfx 1 mg/kg i.v. | 7 | 100** | 86** | 57* | 57* | 29* | 14 |
| PR8 + PB2 (28 as) 20 mg/kg + COATSOME R i.v. | 7 | 100** | 67* | 0 | | | |
| PR8 + PB2 (28 as) 40 mg/kg + COATSOME R i.v. | 6 | 100** | 86** | 29* | 0 | | |
| PR8 + PB2 (28 as) 20 mg/kg + COATSOME Y i.v. | 7 | 100** | 67* | 33* | 0 | | |
| PR8 + PB2 (28 as) 40 mg/kg + COATSOME Y i.v. | 6 | 100** | 86** | 57* | 29* | 0 | |
| PR8 + PB2 (28 as) 20 mg/kg i.v. | 6 | 100** | 50* | 8 | 0 | | |
| PR8 + PB2 (28 as) 40 mg/kg i.v. | 6 | 100** | 100** | 50* | 17 | 0 | |

*P < 0.05
**P < 0.01
***COATSOME R = COATSOME Red label, COATSOME Y = COATSOME Yellow label

Further, in the administration group wherein PB2 (20as) was administered at an amount of 40 mg/kg with 1 mg/kg of Tfx-10, 100% of the mice survived to the 6th day, 75% on the 8th day, 50% to the 9th day, and 25% to the 10th day. Thus, the dose-dependent effect on the prolonging of life was significantly observed. In the administration group wherein only PB2 (20as) was administered at an amount of 40 mg/kg without Tfx-10, a significant effect on the prolonging of life was observed only on the survival rate of the 6th day.

In the administration group wherein PA (20as) was administered at an amount of 20 mg/kg with 5 mg/kg of Tfx-10, a significant effect on the prolonging of life was observed on the 5th and 6th days, but no significant effect on the prolonging of life was observed on or after the 7th day. In the administration group wherein only PA (20as) was administered at an amount of 40 mg/kg without Tfx-10, no significant effect on the prolonging of life was observed.

As to PB2 (28as), in the administration groups wherein PB2 (28as) was administered at an amount of 20 mg/kg or 40 mg/kg with 1 mg/kg of Tfx-10, 57% and 86% of the mice survived to the 6th day, and 29% and 57% to the 8th day. Thus, a significant effect on the prolonging of life was observed. Further, in the administration group wherein PB2 (28as) was administered at an amount of 40 mg/kg with 20 μmol/kg of COATSOME EL-C-01 (COATSOME, Red label in Table 1), 29% of the mice survived even to the 7th day. In the administration group wherein PB2 (28as) was administered at an amount of 40 mg/kg with 20 μmol/kg of COATSOME EL-N-01 (COATSOME, Yellow label in Table 1), 57% of the mice survived to the 7th day. Thus, a significant effect on the prolonging of life was observed. As to the comparison of two COATSOMEs, there is a tendency for COATSOME EL-N-01 (Yellow label; particle size=199±60 nm; weak negatively charged) to exhibit a stronger effect on the prolonging of life than COATSOME EL-C-01 (Red label; particle size=453±150 nm; positively charged).

In the antisense oligonucleotide control groups, i.e., the administration group wherein PB2 (20ran) was administered at an amount of 20 mg/kg with 5 mg/kg of Tfx-10, and the administration group wherein PA (20ran) was administered at an amount of 20 mg/kg with 5 mg/kg of Tfx-10, no significant effect on the prolonging of life was observed.

In the administration group wherein the mice that were not inoculated with the PR8 strain, i.e., non-infected normal mice, were treated, that is, the administration group wherein PB2 (20as) was administered to the non-infected normal mice at an amount of 20 mg/kg with 5 mg/kg of Tfx-10, and the administration group wherein PA (20as) was administered to the non-infected normal mice at an amount of 20 mg/kg with 5 mg/kg of Tfx-10, no effect was observed on the body weight or the survival rate.

Although the results are not shown in FIGS. 5 to 7 or Table 1, experiments wherein N-[1-(2,3-dioleoyloxy)propyl]-N,N,N-trimethylammonium methylsulfate (DOTAP; Boehringer Mannheim) at an amount of 10 mg/kg or 30 mg/kg was administered instead of Tfx-10 were carried out. Any combination of PB2 (20as), PA (20as), or PB2 (28as) therewith did not inhibit the decrease of the body weight or death of the mice. No significant effect on the prolonging of life was observed.

As above, it is apparent that PB2 (20as), PA (20as), or PB2 (28as) inhibits the proliferation of the influenza A virus and enhances an anti-viral effect to alleviate the influenzal symptoms, in an in vivo experiment in the presence of Tfx-10 or COATSOME.

### Example 4

### Successive Intravenous Administration of Antisense Oligonucleotides (2)

Two oligonucleotides prepared in Example 2, i.e., PB2 (20as) and PB2 (20ran) were used in the present Example.

The following cationic liposomes, which are commercially available trasfection-reagents, were used: FuGENE-6 (blended lipid with other compounds in 80% ethanol, Boehringer Mannheim, GmbH, Germany); DOTAP {N-[l-(2,3-dioleoyloxy)propyl]-N,N,N-trimethylammonium methylsulfate, Boehringer Mannheim}; Tfx-10 {N,N,N′,N′-tetramethyl-N,N′-bis(2-hydroxyethyl)-2,3-dioleoyloxy-1,4-butanediammonium iodide, and L-dioleoy-phosphatidyethanolamine (DOPE) in a molar ratio of 1:9 (Promega, Madison, Wis., USA)}; and DMRIE-C (1,2-dimyristyloxy-propyl-3-dimethylhydroxy ethylammonium bromide and cholesterol in a molar ratio of 1:1, Gibco, Gaithersburg, Md., USA).

Two or four milligrams of the oligonucleotides, PB2 (20as) and PB2 (20ran), were mixed with 1 mg of liposomes in 1 ml of PBS at room temperature for 15 minutes, as recommended by the supplier.

Female specific-pathogen-free BALB/c mice, weighing 17 to 19 g (6-weeks-old) each, were obtained from Japan SLC Inc. (Shizuoka, Japan). All mice were kept at constant temperature (25±1° C.) and humidity (55±5%) under conditions of a 12-12 hours light-dark cycle throughout the experimental periods.

Mice were anesthetized by intraperitoneal (i.p.) administration of sodium pentobarbital at 50 mg/kg (Abbott Laboratories, USA) and were inoculated intranasally (i.n.) with 40 μl of a PB8 suspension containing 100LD₅₀s. The mice were pre-treated intravenously (i.v.) with a 0.2 ml volume of liposome-oligonucleotide complexes at 24 and 12 hours before the intranasal (i.n.) challenge of the mouse-adapted influenza A virus (days −1), and were post-treated intravenously (i.v.) twice (9 a.m. and 6 p.m.) daily on days 0 to 4. The complexes of PB2 (20as) and PB2 (20ran) (negative control) were used at doses of either 20 mg/kg or 40 mg/kg, and those of the cationic liposomes were either 1 mg/kg, 5 mg/kg, or 25 mg/kg. Ribavirin (positive control) at 40 mg/kg was also injected intravenously (i.v.) into a group of infected mice twice daily on days −1 to 4. Ribavirin [1-(beta-D-ribofuranosyl)-1,2,4-triazole-3-carboxamide] was provided by Yamasa-Shoyu Co. Ltd (Chiba, Japan).

The observation period was over 14 days postinfection, and the efficacy in terms of the reductions in mortality was assessed. The results are shown in Table 2.

TABLE 2
Effect of oligonucleotide-liposome complexes on mean survival in days and survival rate
in influenza A virus-infected mice

| | Dose | Free | FuGENE-6 | DOTAP | Tfx-10 | | DMRIE-C | | |
|---|---|---|---|---|---|---|---|---|---|
| Compounds | (mg/kg) | (PBS) | 1 mg/kg | 1 mg/kg | 1 mg/kg | 5 mg/kg | 1 mg/kg | 5 mg/kg | 25 mg/kg |
| Control | — | 4.0 ± 0.5 | 4.2 ± 0.5 | 3.7 ± 0.4 | 4.1 ± 0.5 | 3.9 ± 0.4 | 4.2 ± 0.8 | 4.3 ± 0.5 | 4.3 ± 0.5 |
| | | [0] | [0] | [0] | [0] | [0] | [0] | [0] | [0] |
| PB2-as | 20 | 4.5 ± 0.7 | 4.7 ± 0.5* | 5.3 ± 0.5* | 8.1 ± 0.7* | 8.5 ± 0.6* | 9.2 ± 0.8* | 11.7 ± 0.7** | 12.7 ± 0.5** |
| | | [0] | [0] | [0] | [25]* | [27]* | [33]* | [43]* | [57]** |
| | 40 | 5.5 ± 0.5* | 5.7 ± 0.6* | 6.6 ± 0.4* | 9.5 ± 0.6* | 11.1 ± 0.7* | 11.3 ± 0.8* | 12.5 ± 1.2** | 13.1 ± 1.1** |
| | | [0] | [0] | [0] | [25]* | [45]* | [33]* | [55]* | [74]** |
| PB2-ran | 40 | 3.9± 0.4 | 4.8 ± 0.7 | 4.3 ± 0.5 | 3.9 ± 0.4 | 4.1 ± 0.5 | 4.2 ± 0.4 | 4.0 ± 0.6 | 4.7 ± 0.5 |
| | | [0] | [0] | [0] | [0] | [0] | [0] | [0] | [0] |
| Ribavirin | 40 | 13.5 ± 1.3** | | | | | | | |
| | | [85]** | | | | | | | |

Data represent the means ± standard deviations of survival in days. Brackets indicate the percent of survival on the 14th day after the virus infection. *P < 0.05, **P < 0.01, compared to the control group of mice with vehicle treatment (mean survival in days: t-test, survival rate: χ² analysis). N = 6-10 per group.

As shown in Table 2, when mice were infected with 100LD₅₀s of influenza A virus, the control mice treated intravenously (i.v.) with all liposomes showed MSDs of 3.7 to 4.3 days and 0% survival. This concentration of liposome showed no in vivo toxicity. PB2 (20as) at 40 mg/kg significantly prolonged the MSDs of the infected mice. The protective effect of PB2 (20as) was more enhanced when the infected mice were treated intravenously (i.v.) with the complex of liposomes. Especially, DMRIE-C showed the highest efficacy among these liposomes. Mice treated with the complexes of PB2 (20as) (20 mg/kg, 40 mg/kg) and DMRIE-C (25 mg/kg) showed MSDs of 12.7 and 13.1 (p<0.01) days, respectively, and 57% and 74% (p<0.01) survival rates. PB2 (20as)/DMRIE-C complexe showed increased protective activity with an increase in the liposome, DMRIE-C. The Tfx-10 showed a protective activity than DMRIE-C. However, FuGENE-6 and DOTAP showed no protective activity. Furthermore, the liposomally encapsulated control oligonucleotide, PB2 (20ran), at 40 mg/kg had no effect on either the MSDs or the survival. Overdosage of Tfx-10 at 5 mg/kg had toxicity, as manifested by reduced body weights and MSDs in the influenza A virus-infected mice. In contrast, DMRIE-C had no toxicity at a dose of 25 mg/kg and showed more efficacy than Tfx-10. The protective effect of PB2 (20as) in the presence of DMRIE-C was close to that of ribavirin.

To characterize the inhibitory effects of PB2 (20as), it was determined the influenza A virus titers in the lungs of mice treated with PB2 (20as) at 40 mg/kg in the presence of DMRIE-C (5 mg/kg) twice daily for 4 days postinfection. The results are shown in Table 3.

TABLE 3
Efficacy of DMRIE-C encapsulated oligonucleotides against influenza
A virus titers in mouse lung.

| | Dose | Virus titers |
|---|---|---|
| Compounds | (mg/kg) | (log₁₀ TCID₅₀s/g lung tissues) |
| Control | — | 8.55 ± 0.40 |
| PB2-as | 40 | 7.05 ± 0.30*** |
| PB2-ran | 40 | 8.72 ± 0.43 |
| Ribavirin | 40 | 7.11 ± 0.44*** |

TCID₅₀ values determined at six time measurements with the lung homogenate obtained from a mouse. Data represent the means ± standard deviations for six mice. Peak virus titers were detected on the 4th day after i.v. treatment. ***P < 0.001, compared to the control group of mice with vehicle treatment (t-test).

As shown in Table 3, the virus titers in the lungs of the liposomal PB2 (20as)-treated group of mice were 10 or more-fold lower than those in the lungs of the control mice treated with the vehicle of DMRIE-C. The degree of reduction induced by PB2 (20as) was the same as that induced by ribavirin at 40 mg/kg. In the negative control group, treatment with PB2 (20ran) at 40 mg/kg had no influence on the virus titers in the lungs. This result suggests that liposomally encapsulated PB2 (20as) has a specific effect against the influenza A virus growth in lung tissues. The above results indicate that the i.v. administration of PB2 (20as) endocapsulated with DMRIE-C may be useful as a potential therapeutic treatment for influenza A virus disease.

### INDUSTRIAL APPLICABILITY

The pharmaceutical composition for treating or preventing influenza according to the present invention contains the particular antisense oligonucleotide and the particular liposome, and thus, an effective prevention or treatment against the infection with the influenza viruses can be carried out.

Free Text in Sequence Listing

A base sequence of SEQ ID NO: 1 is a base sequence complementary to the base sequence (base sequence of SEQ ID NO: 11 in the Sequence Listing) consisting of 20 bases and containing the translational initiation codon AUG of the PB2 gene of the influenza virus.

A base sequence of SEQ ID NO: 2 is a base sequence complementary to the base sequence (base sequence of SEQ ID NO: 11 in the Sequence Listing) consisting of 20 bases and containing the translational initiation codon AUG of the PB2 gene of the influenza virus; all of the internucleotide bonds being phosphorothioate bonds.

A base sequence of SEQ ID NO: 3 is a random base sequence with respect to the base sequence (base sequence of SEQ ID NO: 11 in the Sequence Listing) consisting of 20 bases and containing the translational initiation codon AUG of the PB2 gene of the influenza virus; all of the internucleotide bonds being phosphorothioate bonds.

A base sequence of SEQ ID NO: 4 is a base sequence complementary to the base sequence (base sequence of SEQ ID NO: 13 in the Sequence Listing) consisting of 20 bases and containing the translational initiation codon AUG of the PA gene of the influenza virus.

A base sequence of SEQ ID NO: 5 is a base sequence complementary to the base sequence (base sequence of SEQ ID NO: 13 in the Sequence Listing) consisting of 20 bases and containing the translational initiation codon AUG of the PA gene of the influenza virus; all of the internucleotide bonds being phosphorothioate bonds.

A base sequence of SEQ ID NO: 6 is a random base sequence with respect to the base sequence (base sequence of SEQ ID NO: 13 in the Sequence Listing) consisting of 20 bases and containing the translational initiation codon AUG of the PA gene of the influenza virus; all of the internucleotide bonds being phosphorothioate bonds.

A base sequence of SEQ ID NO: 7 is a base sequence complementary to the base sequence (base sequence of SEQ ID NO: 12 in the Sequence Listing) consisting of 28 bases and containing the translational initiation codon AUG of the PB2 gene of the influenza virus.

A base sequence of SEQ ID NO: 8 is a base sequence complementary to the base sequence (base sequence of SEQ ID NO: 12 in the Sequence Listing) consisting of 28 bases and containing the translational initiation codon AUG of the PB2 gene of the influenza virus; all of the internucleotide bonds being phosphorothioate bonds.

A base sequence of SEQ ID NO: 9 is a base sequence complementary to the base sequence consisting of 28 bases and containing the translational initiation codon AUG of the PA gene of the influenza virus.

A base sequence of SEQ ID NO: 10 is a base sequence complementary to the base sequence consisting of 28 bases and containing the translational initiation codon AUG of the PA gene of the influenza virus; all of the internucleotide bonds being phosphorothioate bonds.

As above, the present invention was explained with reference to particular embodiments, but modifications and improvements obvious to those skilled in the art are included in the scope of the present invention.

1. A pharmaceutical composition for treating or preventing influenza comprising an oligonucleotide containing a base sequence complementary to a base sequence of a target region containing a translational initiation codon AUG of a PB2 gene or a PA gene, a liposome stable in blood, and a pharmaceutically acceptable carrier or dilute.

2. The pharmaceutical composition for treating or preventing influenza according to claim 1, wherein at least one internucleotide bond in the oligonucleotide is a phosphorothioate bond.

3. The pharmaceutical composition for treating or preventing influenza according to claim 2, wherein the oligonucleotide is an oligonucleotide containing a base sequence of SEQ ID No: 2.

4. The pharmaceutical composition for treating or preventing influenza according to claim 2, wherein the oligonucleotide is an oligonucleotide containing a base sequence of SEQ ID NO: 5.

5. The pharmaceutical composition for treating or preventing influenza according to claim 2, wherein the oligonucleotide is an oligonucleotide containing a base sequence of SEQ ID NO: 8.

6. The pharmaceutical composition for treating or preventing influenza according to claim 1, wherein the liposome is formed from a mixture of N,N,N′,N′-tetramethyl-N,N′-bis(2-hydroxyethyl)-2,3-dioleoyloxy-1,4-butanediammonium iodide and L-dioleoylphosphatydylethanolamine.

7. The pharmaceutical composition for treating or preventing influenza according to claim 1, wherein the liposome is formed from a mixture of L-α-dipalmitoylphosphatydylcholine, cholesterol, and stearylamine or L-α-dipalmitoylphosphatydyl-glycerol.

8. The pharmaceutical composition for treating or preventing influenza according to claim 1, wherein the liposome is formed from a mixture of 1,2-dimyristyloxy-propyl-3-dimethylhydroxy ethylammonium bromide and cholesterol in a molar ratio of 1:1.

9. A method for treating or preventing influenza, comprising administering to a subject in need thereof an oligonucleotide containing a base sequence complementary to a base sequence of a target region containing a translational initiation codon AUG of a PB2 gene or a PA gene, and a liposome stable in blood, in an amount effective in treating or preventing influenza.

10. An oligonucleotide containing a base sequence of SEQ ID NO: 8.

11. A pharmaceutical composition comprising the oligonucleotide according to claim 10 and a pharmaceutically acceptable carrier or dilute.

12. A method for treating or preventing influenza, comprising administering to a subject in need thereof the oligonucleotide according to claim 10 in an amount effective in treating or preventing influenza.

13. An oligonucleotide containing a base sequence of SEQ ID NO: 10.

14. A pharmaceutical composition comprising the oligonucleotide according to claim 13 and a pharmaceutically acceptable carrier or dilute.

15. A method for treating or preventing influenza, comprising administering to a subject in need thereof the oligonucleotide according to claim 13 in an amount effective in treating or preventing influenza.
